(19) United States
(12) Patent Application Publication
(10) Pub. No.: US 2022/0202695 A1
(43) Pub. Date: Jun. 30, 2022

(54) BIOTECHNOLOGICAL PRODUCTION OF FLAVORINGS FROM POMACES OF THE CURRANT, STRAWBERRY, RASPBERRY AND WINE INDUSTRY

(71) Applicant: SYMRISE AG, Holzminden (DE)

(72) Inventors: Holger ZORN, Wettenberg (DE); Svenja SOMMER, Gießen (DE); Nadine SELLA, Pohlheim (DE); Christine SCHLERING, Ochsenfurt (DE); Martin RÜHL, Echzell (DE); Marco FRAATZ, Gießen (DE); Julia BÜTTNER, Gießen (DE); Tobias TRAPP, Holzminden (DE); Gerhard KRAMMER, Holzminden (DE); Torsten GEISSLER, Einbeck (DE)

(21) Appl. No.: 17/611,432

(22) PCT Filed: May 15, 2020
(86) PCT No.: PCT/EP2020/063621

§ 371 Date: Nov. 15, 2021

(30) Foreign Application Priority Data
May 15, 2019 (EP) 19174737.7

(51) Int. Cl. A61K 8/9789; C12P 7/26; C12P 7/04

(52) U.S. Cl. CPC A61K 8/9789; A61K 2800/85; C12P 7/04; C12P 7/26

(57) ABSTRACT

The present invention relates to a biocatalytic process for producing an aroma substance or a mixture of aroma substances, comprising the steps of:
providing a conversion medium comprising a plant component of the gooseber-ry family, the rose family and/or the grapevine family;
contacting the conversion medium with at least one fungus from the division of stander fungi capable of forming an aroma substance or a mixture of aroma sub-stances on the conversion medium;
converting the plant component to the aroma substance or mixture of aroma substances with the aid of the fungus; and optionally
recovering the aroma substance or mixture of aroma substances,
wherein the aroma substance or mixture of aroma substances preferably com-prises at least one compound selected from the group consisting of 2-octanone, 2-nonanone, 2-undecanone, linalool oxides, benzaldehyde, geraniol, 2-octanol, methylanthranilate, 2-aminobenzaldehyde and linalool.

The present invention relates to the field of aroma substances. More particularly, the present invention relates to a method of preparing an aroma substance or a mixture of aroma substances as described herein. The invention further relates to the use of a conversion medium comprising an ingredient based on plant parts of the gooseberry family, rose family or grapevine family as described herein and/or a biocatalyst as described herein for producing an aroma substance or a mixture of aroma substances. Furthermore, the present invention relates to compositions comprising compounds as described herein and the use thereof in a nutritional, cosmetic, hygienic or edible preparation as described herein.

Natural aroma substances are classically obtained by extraction from plants. Linalool may be mentioned here as an example. Linalool is a monohydric alcohol which belongs to the group of terpenes. The compound is colorless, flammable and has a distinct fragrant odor.

Well-known and abundant sources of linalool are herbs such as basil, savory, coriander, oregano and thyme. Linalool can be used for a variety of applications. For example, because of its perfuming and/or deodorizing function, the compound is used as a flavoring agent, in aromatic oils, or in the perfume industry as a substitute for bergamot. Furthermore, the compound can be found as an aroma in wine.

In order to meet the strong and steadily growing demand for linalool, a not insignificant proportion of the market supply is now chemically synthesized. Although chemical syntheses offer the possibility of producing large quantities of flavoring substances, many syntheses are not very environmentally compatible. In particular, the mostly negligible reaction rates under normal conditions mean that acceptable yields can only be achieved by applying harsh synthesis conditions such as high temperatures and high pressures. Furthermore, chemical syntheses not infrequently employ heavy metal catalysts, flammable gases and organic solvents, the use of which is fraught with well-known disadvantages. For this reason, there is a growing need for alternative production processes for linalool, but also for other aroma-active compounds, which at least partially overcome the disadvantages of the known methods.

This could be remedied by biotechnological processes based on the biotransformation of natural precursors into desired aroma substances using microorganisms or their enzymes (Berger, R. G., Flavors and Fragrances: Chemistry, Bioprocessing and Sustainability, (2007), Berlin Heidelberg, Springer Verlag). Agricultural by-products, some of which are rich in dietary fibers and secondary metabolites as potential aroma precursors, could have great potential for the biotechnological production of linalool and other aroma substances.

It was therefore the primary object of the present invention to remedy the disadvantages described above and to provide a process for the preparation of aroma substances such as linalool. In particular, it was a concern to provide a process which is more environmentally friendly than the known chemical syntheses.

WO2013/034613 discloses a method of producing a beverage or beverage base, wherein a medium is fermented in at least one aerobic fermentation process and the medium is fermented by mycelium from at least one basidiomycete.

According to the invention, this primary object is solved by a method for producing an aroma substance or a mixture of aroma substances, comprising the steps:

Providing a conversion medium (also referred to herein in part as a culture medium or substrate) comprising an ingredient based on plant parts of the gooseberry family (Grossulariaceae), rose family (Rosaceae) or grapevine family (Vitaceae); contacting the conversion medium with a biocatalyst; conversion of the ingredient based on plant parts of the gooseberry family, rose family or grapevine family to form the aroma substance or mixture of aroma substances by means of the biocatalyst; and, optionally, recovering the aroma substance or mixture of aroma substances.

Further objects, aspects and preferred embodiments of the present invention will be apparent from the following explanations, the appended examples, the figures and in particular the appended patent claims.

The figures show:

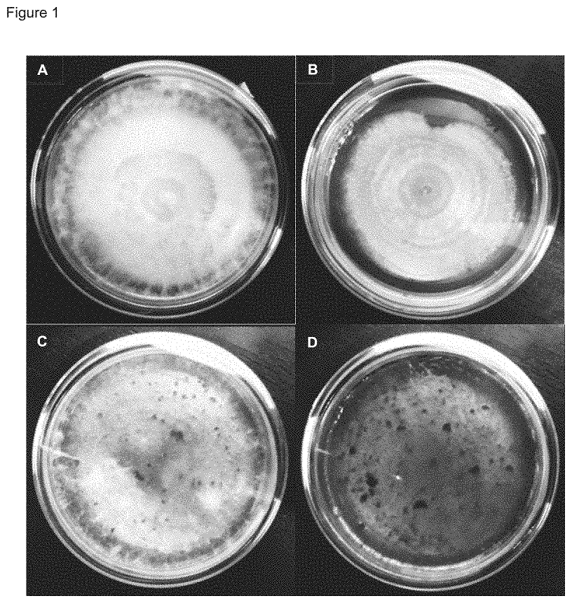

FIG. 1: Comparison of a conversion on a culture medium according to the invention (conversion medium) with a standard culture medium using the example of the fungus W. cocos. Photographs of emersed cultures of W. cocos after 11 days (d) on different culture media are shown. A: Malt extract agar (MEA). B: MEA+0.624% monosodium L-aspartate, C: 3% pomace, D: 3% pomace+0.624% monosodium L-aspartate.

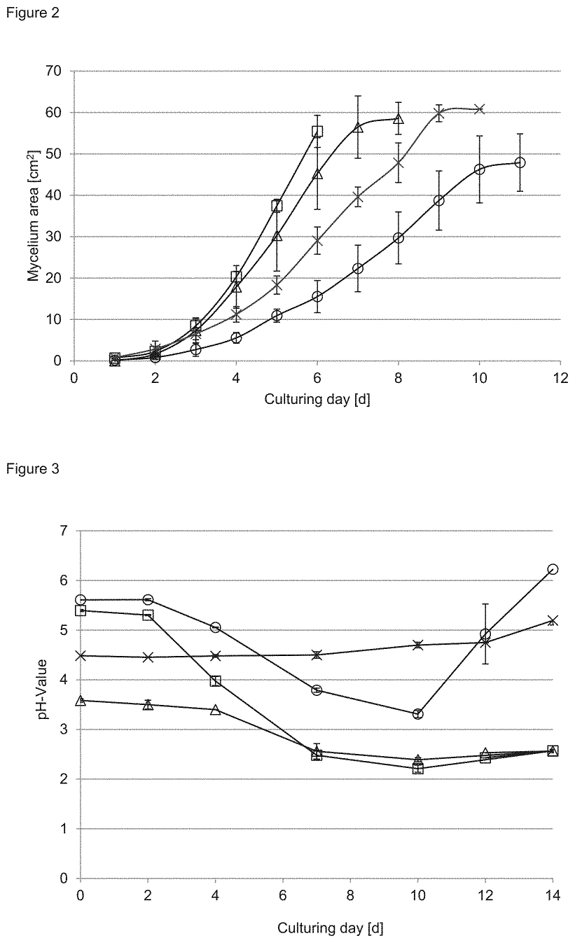

FIG. 2: Comparison of a conversion on culture medium according to the invention with standard culture medium using the example of the fungus W. cocos. Shown are growth curves of the emersed cultures of W. cocos on four different media: MEA (□); MEA+Asp 0; 3TT (x); 3TT+Asp (∘). Abbreviations used: 3TT: 3% pomace of Titania variety, Asp: 0.624% monosodium L-aspartate.

FIG. 3: Comparison of a conversion on culture medium according to the invention with standard culture medium using the example of the fungus W. cocos. Shown is the course of pH-value of the media during emersed cultivation of W. cocos on MEA (□); MEA+Asp (Δ); 3TT (x); 3TT+Asp (o). Abbreviations used as described previously.

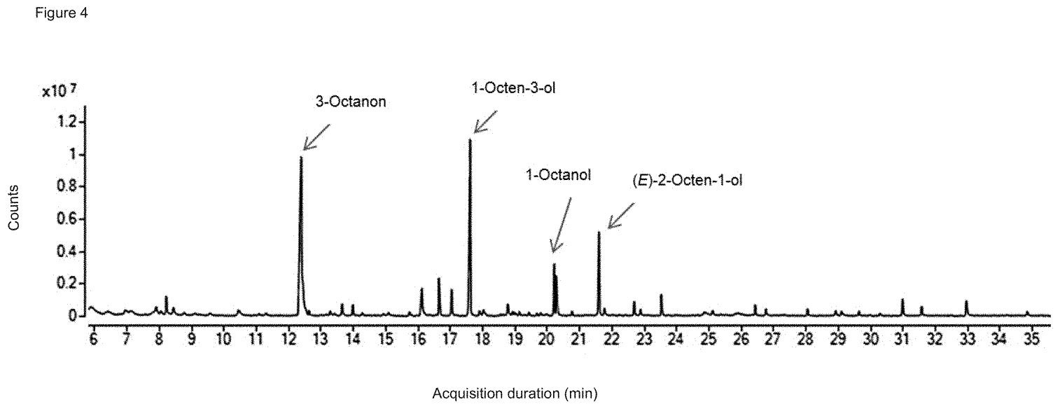

FIG. 4: GC-MS chromatogram (splitless measurement) of a conversion on a culture medium not according to the invention, using the example of a fermentation of W. cocos on MEA. Shown is the chromatogram of a sample taken after 8 days of culture along with substance suggestions.

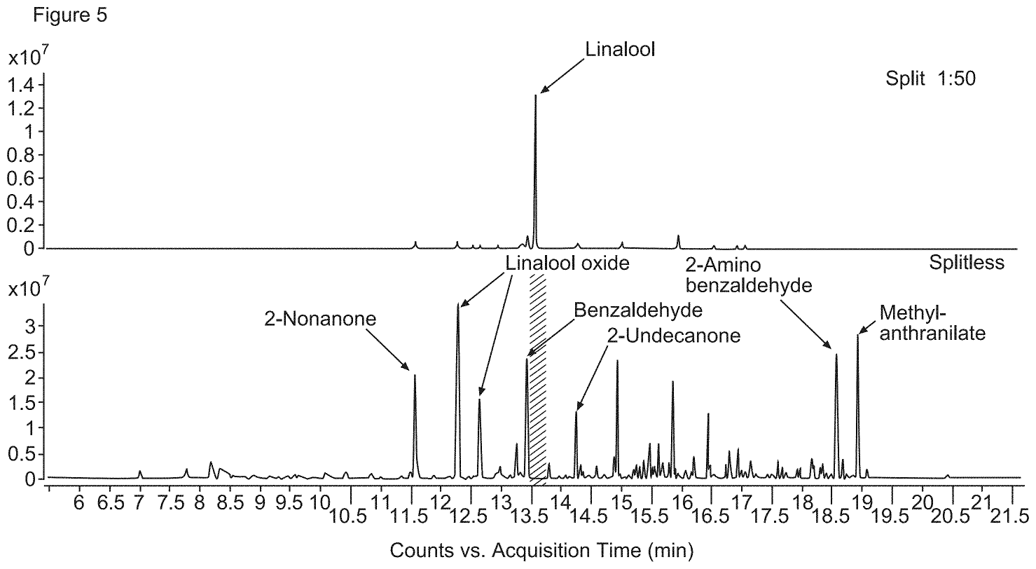

FIG. 5: GC-MS chromatograms of a conversion on culture medium according to the invention using the example of an emersed fermentation of W. cocos on 3% redcurrant pomace, 0.624% monosodium L-aspartate, 3% agar-agar. Shown is the chromatogram of a sample drawn after 11 days of culture; top: Measurement with a split of 50:1, below splitless, where the MS detector was switched off between 13.5 min-13.7 min (grey background).

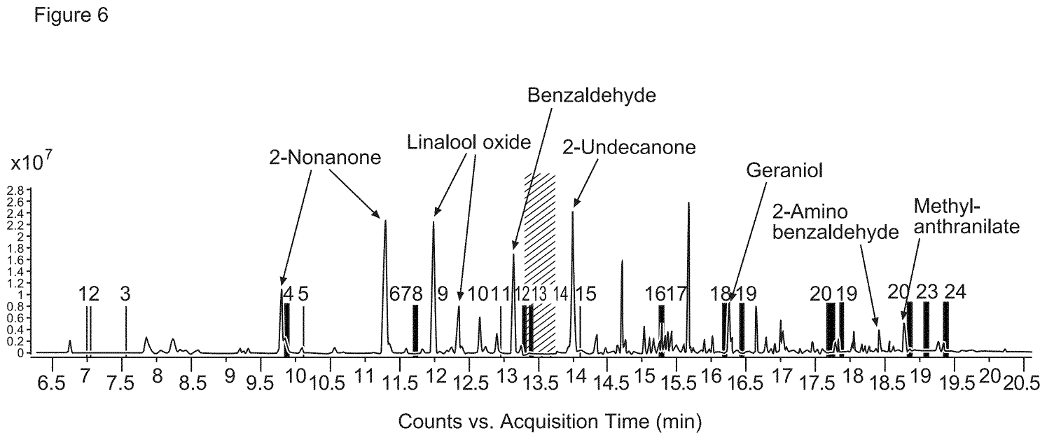

FIG. 6: GC chromatogram with ODP trace and substance suggestions of a conversion on culture medium according to the invention using the example of an emersed fermentation of W. cocos on 3% redcurrant pomace and 0.624% monosodium L-aspartate. Shown is the chromatogram of a sample drawn after 10 days of culture; between 13.2 min and 13.7 min, the MS detector was deactivated (gray background). Black bands with numbers typically indicate odors to be expected according to the following overview.

| | |
|---|---|
| 1 | green |
| 2 | cheese |
| 3 | solvent |
| 4 | sweetish, green |
| 5 | fungal, champignon |
| 6 | green, sweetish, flowery |
| 7 | fungal |
| 8 | green, flowery |
| 9 | dull, green, fir, flowery |
| 10 | dull |
| 11 | dull, green, flowery |
| 12 | marzipan, sweetish |
| 13 | floral, citrus, fresh |
| 14 | green |
| 15 | sweetish, flowery |
| 16 | green, fresh, citrus |
| 17 | green, fir |
| 18 | Fir, flowery |
| 19 | solvent, sweetish |
| 20 | dull, nasty, fried |
| 21 | caramel |
| 22 | fruity |
| 23 | dull, green |
| 24 | green |

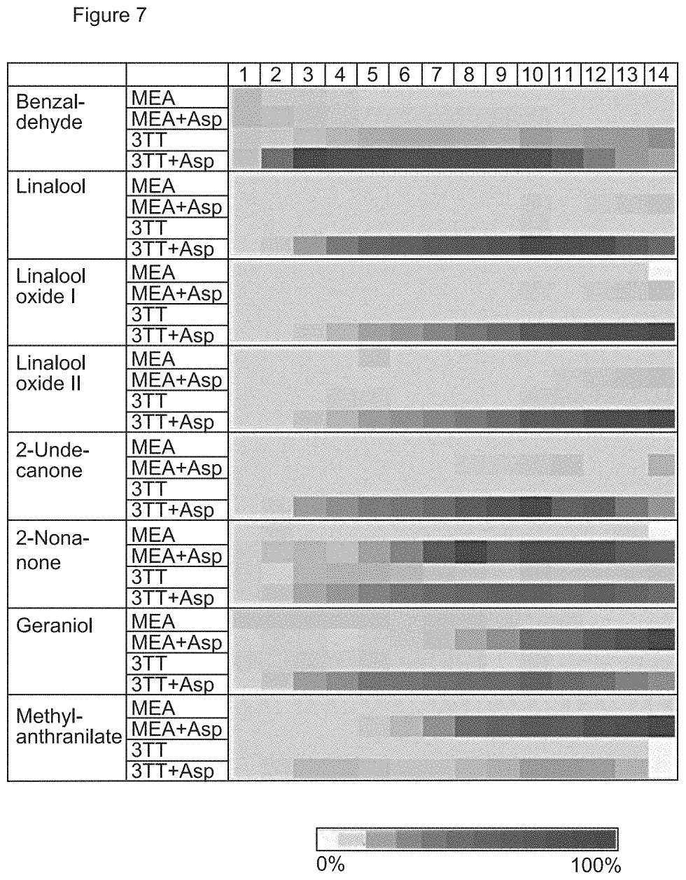

FIG. 7: Heatmap of selected aromas during cultivation of W. cocos on different media; relative change during 14 days of culture. The peak areas of EIC at m/z (benzaldehyde)=106, m/z (linalool)=93, m/z (linalool oxide I, II)=111, m/z (2-undecanone)=71, m/z (2-nonanone)=58, m/z (geraniol)=69, m/z (methyl anthranilate)=119 served as a basis. The assignment of the respective saturation level of a selected fragment was done according to the scheme shown below based on the relative peak area of EIC (0% to 100%) of the selected fragment at the respective time point (1 to 14 days).

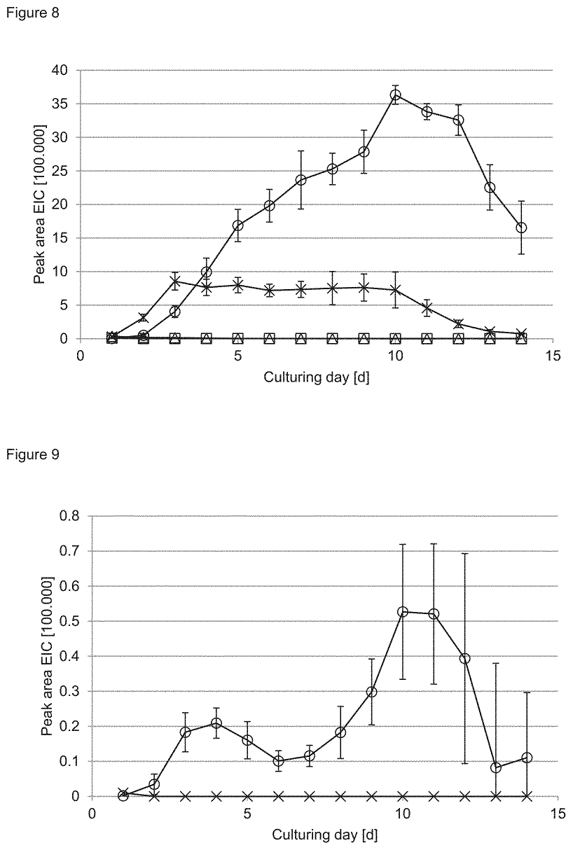

FIG. 8: Development of the aroma profile of linalool and benzaldehyde during the conversion on culture medium according to the invention in comparison to a corresponding conversion on standard culture medium using the example of a fermentation of W. cocos. Evolution of linalool on MEA (□), evolution of linalool on 3TT+Asp (∘); evolution of benzaldehyde on MEA (Δ); evolution of benzaldehyde on 3TT+Asp (x). For the preparation of the EIC, the fragment at m/z=93 was chosen for linalool, and m/z=106 for benzaldehyde. Abbreviations used as previously described.

FIG. 9: Development of the aroma profile of methyl anthranilate (m/z=119) during the conversion on culture medium according to the invention in comparison to a corresponding conversion on standard culture medium using the example of a fermentation of W. cocos. Development of methylanthranilate on MEA (x); development of methylanthranilate on 3TT+Asp (∘).

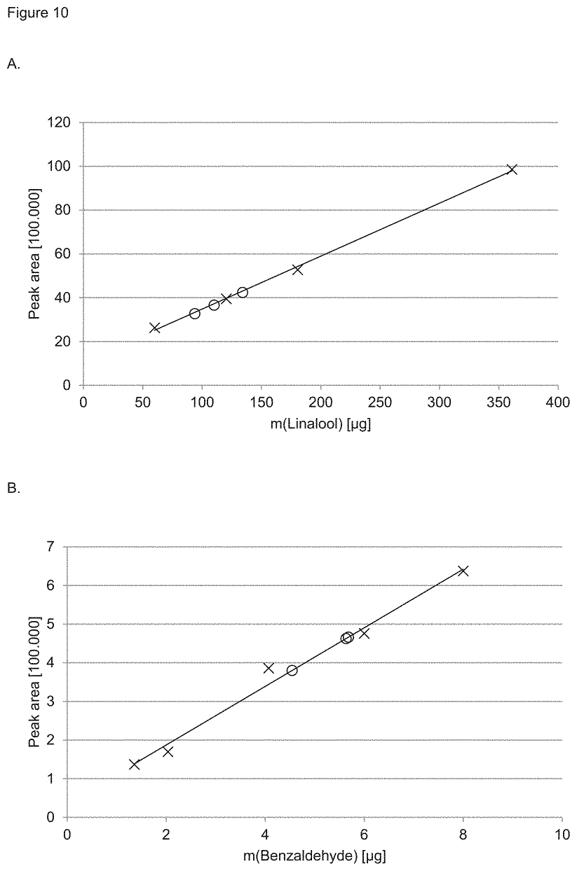

FIG. 10: Semiquantitative estimation of the concentrations of selected aroma substances (∘) after conversion on culture medium according to the invention, using the example of a fermentation of W. cocos on 3% pomace with 0.624% monosodium L-aspartate. Here, the headspace of the cultures was analyzed after 10 d and compared with a calibration series (x) determined by means of standards. A. linalool; B: benzaldehyde; C: methyl anthranilate. The estimated amount is plotted in μg per plate.

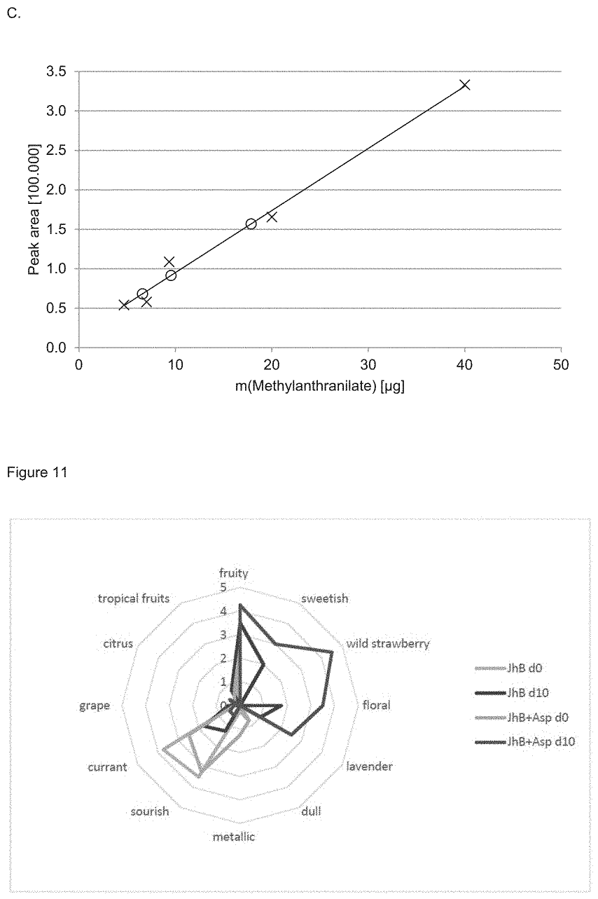

FIG. 11: Sensory evaluation of a conversion on a culture medium according to the invention using the example of an emersed fermentation of W. cocos on 3% redcurrant pomace. Evaluation of given attributes on a scale from 0 to 5 by four trained persons. Abbreviations used: JhB: currant pomace, Asp: 0.624% monosodium L-aspartate, d0: non-cultivated reference medium, d10: cultivation with W. cocos for 10 days.

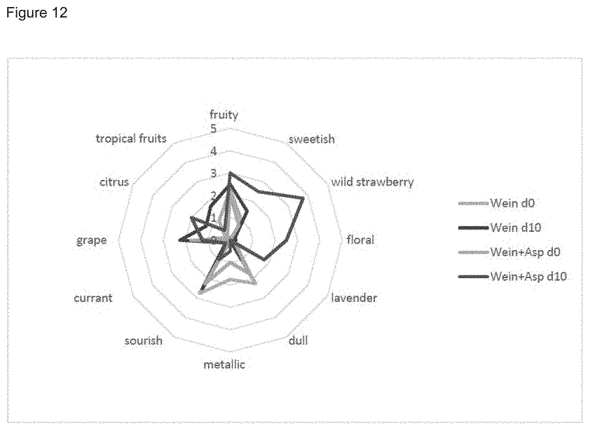

FIG. 12: Sensory evaluation of a conversion on a culture medium according to the invention using the example of an emersed fermentation of W. cocos on 3% grape pomace of the Miller-Thurgau variety. Evaluation of given attributes on a scale from 0 to 5 by four trained persons. Abbreviations used: Wine: grape pomace, Asp: 0.624% monosodium L-aspartate, d0: non-cultivated reference medium, d10: cultivation with W. cocos for 10 days.

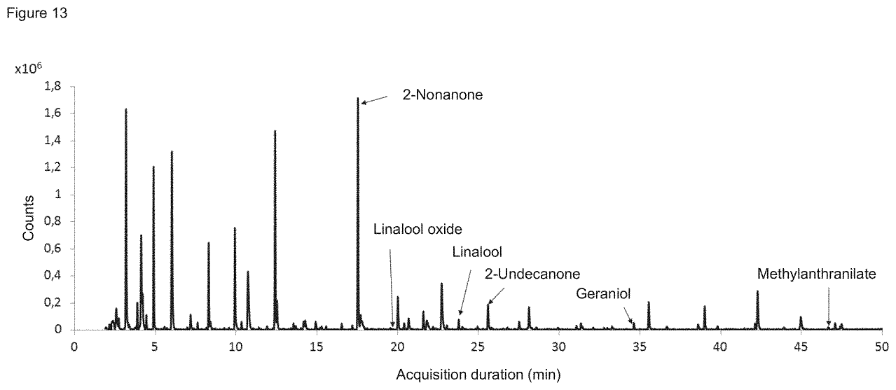

FIG. 13: GC-MS chromatograms of a conversion on culture medium according to the invention, using the example of an emersed fermentation of W. cocos on 3% grape pomace of the Muscaris variety, 0.6% monosodium L-aspartate, 1.5% agar-agar. Shown is the chromatogram of a sample taken after 10 days of culture; measurement with a split of 3:1.

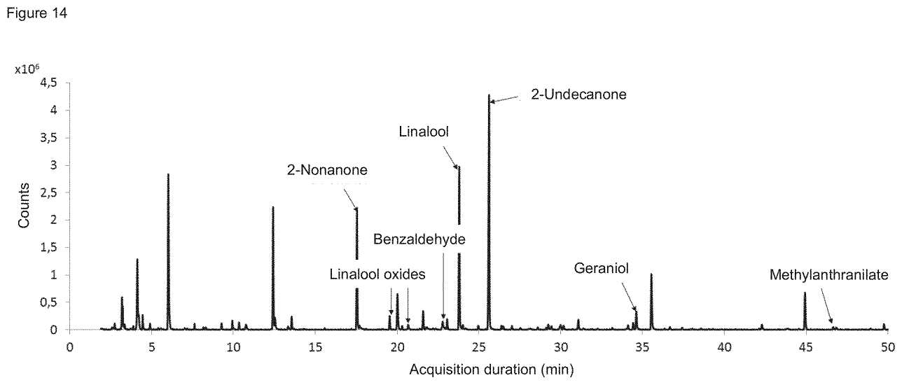

FIG. 14: GC-MS chromatograms of a conversion on culture medium according to the invention using the example of an emers fermentation of W. cocos on 3% strawberry puree residue, 0.6% monosodium L-aspartate, 1.5% agar-agar. Shown is the chromatogram of a sample taken after 8 days of culture; measurement with a split of 3:1.

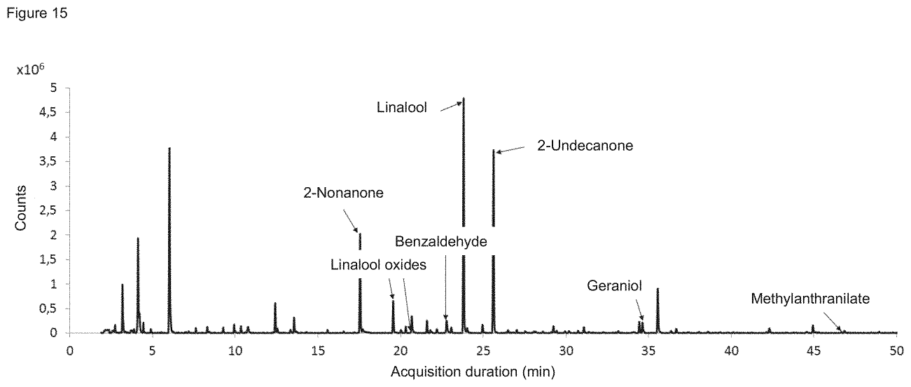

FIG. 15: GC-MS chromatograms of a conversion on culture medium according to the invention using the example of an emersed fermentation of W. cocos on 3% raspberry puree residue, 0.6% monosodium L-aspartate, 1.5% agar-agar. Shown is the chromatogram of a sample taken after 8 days of culture; measurement with a split of 3:1.

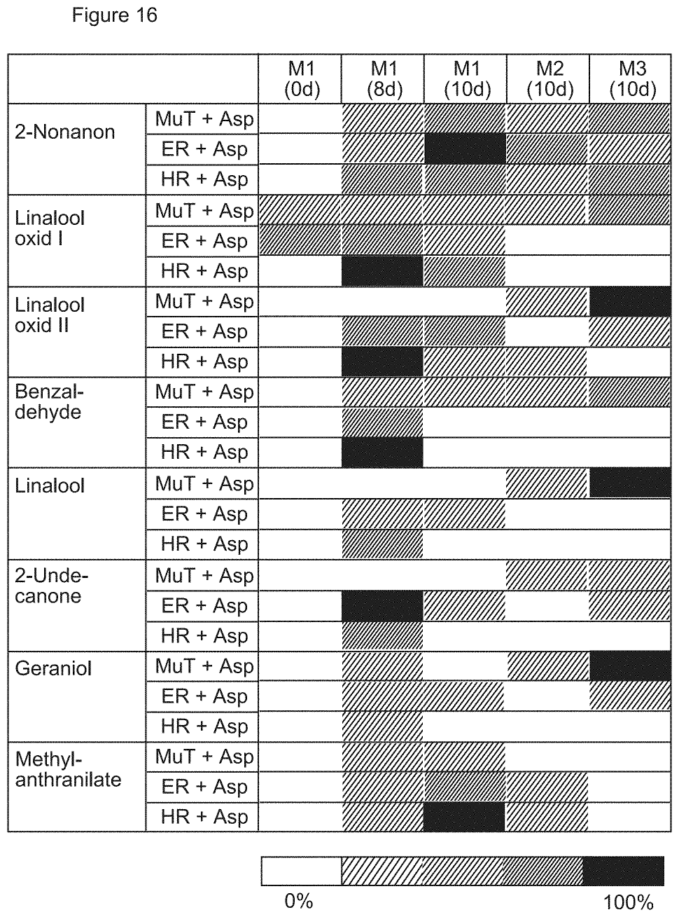

FIG. 16: Heatmap of selected aromas during the cultivation of W. cocos on different culture media and different culture times. The peak areas of the EIC at m/z (benzaldehyde)=106, m/z (linalool)=93, m/z (linalool oxide I, II)=111, m/z (2-undecanone)=71, m/z (2-nonanone)=58, m/z (geraniol)=69, m/z (methyl anthranilate)=119 served as a basis. The assignment of the respective saturation level of a selected fragment was based on the relative peak area of the EIC (0% to 100%) of the selected fragment according to the scheme shown below. Emersed culture media used: M1: 3% pomace, M2: 3% pomace and 0.4% glucose. M3: 3% pomace and 0.8% glucose. Cultivation time: 0 d: non-cultured reference medium, 8 d: 8 days, 10 d: 10 days. Abbreviations used: ER: non-varietal strawberry puree residue, HR: non-varietal raspberry puree residue, MuT: Muscaris variety wine pomace, Asp: 0.6% monosodium L-aspartate.

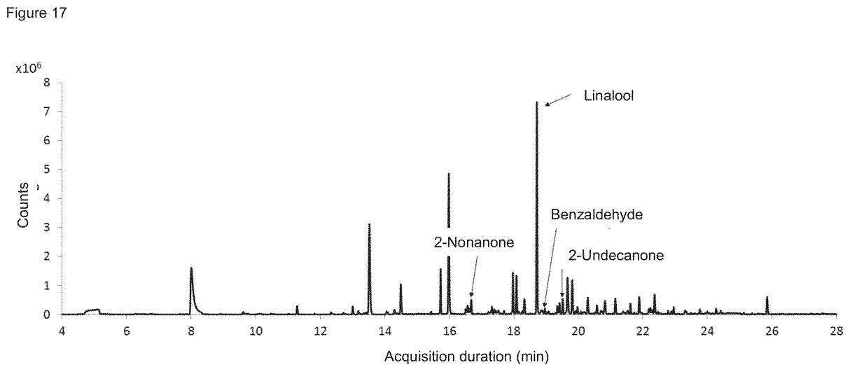

FIG. 17: GC-MS chromatograms of a conversion on culture medium according to the invention using the example of a fixed-bed culture of W. cocos on strawberry puree residue. Shown is the chromatogram of a sample taken after 28 days of culture; measurement by SPME-GC-MS in splitless mode.

The invention is substantially based on the finding that a conversion medium provided with an ingredient based on plant parts of the gooseberry family, rose family or grapevine family contains extremely interesting aroma precursors which can be biocatalytically converted to aroma substances. The term aroma substance is used herein to describe a compound which, in an aroma-active amount, imparts a perceptible taste or odor. In this context, the term “aroma-active” refers to the amount of the compound that is sufficient to elicit a sensory effect at olfactory and/or gustatory receptors when a preparation containing the compound is used. Such an effect may also manifest itself by reducing or masking an unpleasant taste and/or odor based sensory perception.

The method according to the invention avoids the need for harsh manufacturing conditions, organic solvents or heavy metal catalysts, which are hardly avoidable in the case of chemical syntheses. The present invention thus provides an environmentally friendly alternative to chemical syntheses, which moreover meets the growing demand for aroma substances such as linalool.

Furthermore, the method according to the invention is characterized by the fact that it offers the potential to use agricultural waste streams such as leaves or pomace of the gooseberry family, rose family or grapevine family as a source of high-quality aroma substances and thus to be profitably integrated into the value chain of agricultural productions. For this reason, a process is particularly preferred in which the ingredient based on plant parts of the gooseberry family, rose family or grapevine family is provided in the form of leaves and/or a pomace as described herein, and/or an extract thereof. Very particularly preferred is the use of a pomace of gooseberry and in particular a pomace of blackcurrant as described herein. The proportion of the ingredient based on plant parts of the gooseberry family, rose family or grapevine family in methods according to the invention as described herein may generally range from 0.2% by weight to 100% by weight based on the total weight of the conversion medium. If the cultivation is carried out as emersed or submerged cultivation, the proportion of the ingredient based on plant parts of the gooseberry family, rose family or grapevine family in methods according to the invention as described herein is preferably 0.2% by weight to 20% by weight, preferably 0.5% by weight to 10% by weight, further preferably 1% by weight to 5% by weight and most preferably 2% by weight to 4% by weight based on the total weight of the conversion medium. If the cultivation is carried out in the form of a fixed-bed cultivation, the proportion of the ingredient based on plant parts of the gooseberry family, rose family or grapevine family in processes according to the invention as described herein is preferably 10% by weight to 100% by weight, preferably 30% by weight to 90% by weight, further preferably 50% by weight to 80% by weight and most preferably 60% by weight to 70% by weight based on the total weight of the conversion medium.

The term “gooseberry family” refers to plants of the family Grossulariaceae. Preferred according to the invention are methods using plant parts of species and varieties suitable for soft fruit production, in particular species of currants, gooseberries and crosses thereof such as josta. Further preferred are methods using plant parts of species of white or red gooseberries, red, white, black or jet-black currants and crosses and varieties thereof. Most preferably, methods according to the invention use plant parts of blackcurrants (Ribes nigrum) and their varieties and cultivars.

The term “rose family” refers to plants of the Rosaceae family. In accordance with the invention, methods are preferred which use plant parts of species and varieties suitable for soft fruit production, in particular species of strawberries and raspberries and their crosses and varieties.

The term “grapevine family” refers to plants of the family Vitaceae. According to the invention, methods are preferred which use plant parts of species and varieties suitable for soft fruit and wine production, in particular species of grapevine and crosses and varieties thereof. Further preferred are methods using plant parts of species of the noble vine (Vitis vinifera) as well as varieties thereof.

In particular, plant components comprise above-ground vegetative or reproductive tissues, preferably leaves, buds, leaf and/or flower buds, berry fruits and their sub-components such as kernel, peel and pulp. Preferably, the plant parts are leaves and/or berry fruits and further preferably ripe berry fruits as a whole.

An ingredient based on plant parts means in the present case an ingredient obtained from parts of the plant in question. In this context, the ingredient used in methods according to the invention does not necessarily have to occur in nature in an identical manner. Rather, an ingredient of the plant in question may also be obtained by further processing naturally occurring ingredients. Preferred measures of further processing include (partial) drying, (partial) fermentation and/or (partial) pressing. Preferably, the plant is a waste product from other industries using the respective plants or at least parts thereof as raw material. Particularly preferably, the plant parts are berry fruits (as a whole) or residues from juice extraction (so-called pomace).

Of particular interest to the present invention are taste and/or odor impressions that are perceived as pleasant. The assessment of whether a taste and/or odor impression is considered pleasant or rather unpleasant can be made by sensory analysis by a trained panel based on an evaluation of the sensory impression between negative (pleasant) and positive (unpleasant). Further levels such as very negative, neutral and very positive can be provided for more precise classification. The determination of the notes of an aroma substance to be assessed, which is present in a mixture of further compounds, possibly further aroma substances, can be carried out, for example, by means of gas chromatography-olfactometry. In the present case, the aroma substance or the aroma substances are in particular those which impart a pleasant odor impression and can therefore also be referred to as odoriferous substances.

In the context of the preparation of aroma substances with pleasant taste and/or odor impressions, a conversion medium containing an ingredient based on plant parts of the blackcurrant, preferably on plant parts of the blackcurrant (Ribes nigrum) and in particular on plant parts of the blackcurrant of the Titania variety, stands out in particular. This ingredient apparently contains aroma precursors from which aroma substances which are perceived as extremely pleasant can be produced by biocatalytic conversion. The conversion medium provided in the method according to the invention therefore preferably contains an ingredient based on plant parts of the currant, the currant preferably being the black currant and further preferably the black currant of the Titania variety. The aroma substances which can be produced thereby by means of suitable biocatalysts have floral, fresh, fruity notes or notes reminiscent of wild berries or citrus. These aromas are not perceptible when appropriately converted on a standard culture medium based on malt extract. On the contrary, the olfactory impression was described here as fungal, acidic and smelling of tropical fruits. Further advantages result from measures described below.

Preferably, the step of recovering the aroma substance or mixture of aroma substances comprises at least partially separating the produced aroma substance or at least one aroma substance of the mixture of aroma substances from the biocatalyst. Preferably, substantially complete separation from the biocatalyst is performed. Furthermore, the step of recovering may comprise enriching, concentrating and/or isolating the produced aroma substance or at least one aroma substance of the mixture of aroma substances. As used herein, the term “at least one aroma substance” optionally means 1, 2, 3, 4, 5, 6, 7, 8, 9, or aroma substances.

For the purposes of the present invention, whole cell-based biocatalysts have been proven particularly useful due to their ability to reproduce simultaneously or sequentially to the catalyzed conversion reaction. Thus, the step of converting may include culturing in the sense of “growing” or reproducing the biocatalyst. However, it is widely known to those skilled person in the art that conversion using whole cells ultimately relies on one or more catalyzed reactions, so that the present invention also includes methods in which the biocatalyst used to produce the aroma substance or mixture of aroma substances also includes only the catalytically relevant unit of the whole cell-based biocatalyst used to produce the aroma substance or mixture of aroma substances.

The biocatalyst is preferably a fungus, a fungal cell thereof or at least a catalytically relevant unit thereof. A catalytically relevant unit here refers to the macromolecules required for the conversion, such as enzymes or ribozymes, and optionally cofactors and cosubstrates. Preferably, it is a fungus (or a corresponding fungal cell thereof) capable of growing on lignocellulose and/or is/are selected from the group of edible fungi. The ability to grow on lignocellulose is therefore advantageous, as such fungi are particularly well suited for cultivation on an ingredient based on gooseberry as described herein. In addition, aroma substances such as benzaldehyde could result directly from the degradation of lignocellulose. An edible fungus is advantageous in terms of safety in the use of the produced aroma substance or mixture of aroma substances. This preferred requirement profile applies to many fungi from the division (phylum) of the stander fungi (Basidiomycota). Moreover, particularly good odor impressions can be obtained with fungi from the division of the stander fungi, especially if the gooseberry is a black currant as described herein.

Particularly noteworthy is the finding that the conversion with species belonging to the stander fungi leads to an odor that is perceived as pleasant in the overall impression. This means that fungal (mushroomy), sour and tropical fruit smelling notes, which are known as typical fungal aromas, are not or hardly formed. Accordingly, methods are preferred in which the fungus is selected from the division of the stander fungi and/or the plant parts of the gooseberry family are selected from the group of plant parts of the currants, preferably those of the black currants, further preferably those of the black currants of the Titania variety.

In particular, methods as described herein are preferred, wherein the fungus of the division of stander fungi is selected from the group consisting of A. campestris, A. aegerita, A. melea, B. adusta, C. comatus, C. limbatus, F. velutipes, G. odoratum, H. fasciculare, I. consors, L. sulphureus, L. edodes, L. nuda, L. pyriforme, M. cohortalis, M. pseudocorticola, M. scorodonius, P. serotinus, P. chrysosporium, P. flabellatus, P. sapidus, S. crispa, S. hirsutum, T. suaveolens, T. chioneus and W. cocos. In terms of their ability to form aroma substances with pleasant odor impressions, these species are clearly superior to a large number of other fungi or fungus-conversion medium combinations that have been tested and are not specified here.

Further preferred are methods as described herein, wherein the fungus is selected from the group consisting of A. campestris, A. aegerita, C. comatus, G. odoratum, H. fasciculare. I. consors, L. sulphureus. L. edodes, L. pyriforme, M. cohortalis, M. pseudocorticola, M. scorodonius, P. serotinus, P. chrysosporium, P. flabellatus, P. sapidus, S. crispa, T. suaveolens, T. chioneus and W. cocos. This selection leads to a particularly pleasant aroma in at least one possible cultivation form.

Further preferred are methods as described herein, wherein the fungus is selected from the group consisting of A. aegerita, C. comatus, G. odoratum, H. fasciculare, L. sulphureus, M. pseudocorticola, M. scorodonius, P. flabellatus, P. sapidus, S. cispa, T. suaveolens and W. cocos. The above selection leads to a particularly pleasant aroma in at least two possible cultivation forms.

Further preferred are methods as described herein, wherein the fungus is selected from the group consisting of W. cocos and G. odoratum. The fungi G. odoratum and W. cocos show very intense odor impressions on all tested substrates in all tested cultivation forms. G. odoratum produces here a citrus-like aroma, the aroma of W. cocos is floral, fruity and reminiscent of wild strawberries. This selection therefore leads to a particularly pleasant aroma, regardless of the possible cultivation form.

As described above, the preferred biocatalysts include single cells or catalytically active units of the above preferred and further preferred fungi.

Furthermore, according to the invention, methods are preferred in which the conversion medium provided comprises an aspartate source, preferably an L-aspartate source, further preferably a sodium L-aspartate salt and most preferably monosodium L-aspartate. Thus, in the course of the experiments conducted, it was found that the addition of monosodium L-aspartate counteracts an otherwise observed drop in pH and ultimately leads to a particularly intense odor of wild strawberries. After about 10 days of cultivation, the odor intensifies with floral notes and an intense fruity aroma reminiscent of wild strawberries. The signal intensities of the aromas are significantly higher when cultivated on the aspartate-supplemented conversion medium as described herein than when cultivated on standard culture medium based on malt extract. The intensities of linalool are particularly high, which correlates well with the floral odor of the culture. Some aroma-active substances, such as benzaldehyde, linalool, linalool oxide I, II and 2-undecanone are particularly well formed on the aspartate-containing conversion medium, which is preferably provided in the form of a pomace as described herein. The content of the aspartate source can generally range from 0.1% to 20.0% based on the total weight of the conversion medium. If the cultivation is in the form of emersed or submerged cultivation, the content of the aspartate source in methods according to the invention as described herein is preferably from 0.1% by weight to 5% by weight, more preferably from 0.2% by weight to 3% by weight, further preferably from 0.3% by weight to 2% by weight and most preferably from 0.4% by weight to 1% by weight based on the total weight of the conversion medium. If the cultivation is in the form of a fixed bed cultivation, the content of the aspartate source in methods according to the invention as described herein is preferably 0.1% by weight to 20% by weight, more preferably 2.5% by weight to 17.5% by weight, further preferably 5% by weight to 15% by weight and most preferably 7.5% by weight to 12.5% by weight based on the total weight of the conversion medium.

The pH drop can alternatively be counteracted with other buffering substances. Preferably, the pH of the conversion medium during the conversion is within a range of pH 2 to pH 7, preferably pH 4 to pH 6 and in particular pH 4.2 to pH 5.5. In the case of a conversion lasting for several days, this means that the pH range is at least substantially maintained over the entire period.

Additional nutrient sources may be added to the conversion medium to optimize the aroma profile and intensity. In addition to the aspartate source as described herein, these include in particular glucose, trace elements selected from the group consisting of Mg²⁺, Zn²⁺, Fe³⁺, Mn²⁺, Cu²⁺, K⁺, Cl⁻, SO₄²⁻ and PO₄³⁻ and other amino acids.

In the context of the present invention, basically all forms of cultivation are envisaged, even though the specific form may have an influence on the aroma substances formed. For example, particularly good results are obtained with conversion using emersed culture, especially when W. cocos and/or pomace is/are used as the respective biocatalyst and ingredient based on gooseberry family as described herein. Depending on the fungal species used as described herein and the specific form of the ingredient based on the gooseberry family, rose family or grapevine family as described herein, submerged and fixed bed culture also leads to very pleasant aromas.

Particularly advantageous with respect to the producible aroma substances has been found to be a conversion of a conversion medium containing plant parts of the currant as described herein with addition of an aspartate source as described herein by means of a stander fungus as described herein. Instead of an aspartate source, a pH buffering component may also be added to the conversion medium as described hereinabove to maintain the pH within the pH range described herein over the duration of the conversion. In particular, cultivation of W. cocos on currant pomace of the Titania variety provides a floral and fruity odor reminiscent of wild strawberry. W. cocos is an edible fungus that is also used in traditional Chinese medicine. Besides numerous minor components, linalool, benzaldehyde and methylanthranilate are formed as key aroma components.

The method according to the invention is particularly suitable for the production of a aroma substance or a mixture of aroma substances comprising at least 1, preferably 2 or 3, more preferably 4 or 5 and most preferably 6, 7, 8, 9 or 10 compounds selected from 2-octanone, 2-nonanone, 2-undecanone, linalool oxides, benzaldehyde, geraniol, 2-octanol, methylanthranilate, linalool and 2-aminobenzaldehyde. Particularly preferred are methods which deal with the production of at least 1 compound, preferably 2 and further preferably all compounds of linalool, benzaldehyde and methyl anthranilate. These compounds are particularly characteristic of the mixture of aroma substances producible with the conversion medium as described herein. In particular, methyl anthranilate exhibits an intensely fruity odor of wild strawberries.

The exact composition in type and intensity of the aroma substances produced (also referred to herein as aroma composition) can thereby be influenced by varying the time of recovering the aroma substance or mixture of aroma substances. In this context, harvesting times in methods according to the invention have proved useful in which the extraction of the aroma substance or mixture of aroma substances takes place 3 to 14 days, preferably to 14 days and in particular 9 to 13 days after the start of contacting. Apart from this, it is also envisaged to determine the time of extraction of the aroma substance or the mixture of aroma substances as a function of a predetermined aroma composition. This makes it possible, among other things, to harvest at a time at which a particularly pleasant mixture of aroma substances is present and/or a particularly pleasant aroma substance clearly predominates over possibly further aroma substances formed.

Further, the present invention relates to the use of a conversion medium comprising an ingredient based on a plant part of the gooseberry family as described herein and/or a biocatalyst as described herein for producing an aroma substance or mixture of aroma substances as described herein. In particular, the ingredient based on a plant part of the gooseberry family is an ingredient based on plant parts of blackcurrant, such as leaves or pomace thereof as described herein. In particular, the biocatalyst is a fungus from the division of stander fungi as described herein.

Furthermore, the present invention relates to a composition, in particular prepared (preparable) according to the method as described herein, comprising 2 and more particularly all compounds selected from the group consisting of linalool, benzaldehyde and methyl anthranilate. Compositions according to the invention having a volume weight ratio of linalool to benzaldehyde between 100:1 and 4:1 and/or a volume weight ratio of linalool to methyl anthranilate between 50:1 and 2:1 and/or a volume weight ratio of benzaldehyde to methyl anthranilate between 3:1 and 1:10 are preferred. Compositions according to the invention further preferably comprise at least one other compound, preferably 2 or 3, more preferably 4 or 5 and most preferably 6, 7, 8, 9 or 10 compounds selected from the group consisting of 2-octanone, 2-nonanone, 2-undecanone, linalool oxides, benzaldehyde, geraniol, 2-octanol, methylanthranilate, 2-aminobenzaldehyde and linalool.

Furthermore, the present invention relates to the use of compositions according to the invention as described herein as or in a nutritional, cosmetic, hygienic or edible preparation, in particular as a mixture of aroma substances (preferably for the purposes described herein). Accordingly, the present invention also relates to nutritional, cosmetic, hygienic or edible preparations comprising or consisting of a composition according to the invention as described herein.

The invention is explained in more detail below with reference to the following examples.

### EXAMPLES

1. Experimental Part

1.1 Materials and Chemicals The side streams of the currant industry and wine industry used were provided by Geisenheim University, Germany. The raspberry or strawberry puree residues were prepared by pureeing and passing fresh, commercially available, non-varietal berry fruits. The remaining puree residues were used as media components. The sources of the fungi used from the division of Basidiomycota are shown in Table 1.

TABLE 1
Overview of the tested fungi, their origin and cultivation medium;
DSMZ: Leibniz Institute DSMZ-German Collection of Microorganisms
and Cell Cultures, Braunschweig, Germany; CBS: CBS-KNAW culture
collection, Westerdijk Fungal Biodiversity Institute, Utrecht,
The Netherlands; S: Collection Institute of Food Chemistry and
Food Biotechnology, Justus Liebig University Giessen; MEA: Malt
extract agar; SNL: Standard nutrient solution agar.

| Name | Strain no. | Origin | Medium |
|---|---|---|---|
| Agaricus campestris | FP44 | S | SNL |
| Agrocybe aegerita | AAE3 | S | MEA |
| Armillaria melea | 2941 | DSMZ | MEA |
| Bjerkandera adusta | 4708 | DSMZ | MEA |
| Coprinus comatus | FP91 | S | MEA |
| Cyathus limbatus | 335.81 | CBS | MEA |
| Flammulina velutipes | 1658 | DSMZ | MEA |
| Gloeophyllum odoratum | 12011 | DSMZ | MEA |
| Hypholoma fasciculare | 2902 | DSMZ | MEA |
| Irpex consors | 7382 | DSMZ | MEA |
| Laetiporus sulphureus | 1014 | DSMZ | MEA |
| Lentinula edodes | 389.89 | CBS | MEA |
| Lepista nuda | 3347 | DSMZ | MEA |
| Lycoperdon pyriforme | 8676 | DSMZ | MEA |
| Marasmius cohortalis | 8257 | DSMZ | MEA |
| Mycena pseudocorticola | 108 | S | MEA |
| Mycetinis scorodonius | 137.83 | CBS | MEA |
| Panellus serotinus | 5204 | DSMZ | MEA |
| Phanerochaete chrysosporium | 481.73 | CBS | MEA |
| Pleurotus flabellatus | 8299 | DSMZ | MEA |
| Pleurotus sapidus | 8266 | DSMZ | MEA |
| Sparassis crispa | FP32 | S | MEA |
| Stereum hirsutum | Bda11 | S | MEA |
| Trametes suaveolens | 5237 | DSMZ | MEA |
| Tyromyces chioneus | 5242 | DSMZ | MEA |
| Wolfiporia cocos | 279.55 | CBS | MEA |

1.2 Strain Maintenance

The fungi to be studied (Table 1) were cultured on malt extract agar plates (MEA) or standard nutrient solution (SNL) agar plates for strain maintenance. All media were steam sterilized at 121° C. for 20 min. The media were composed as follows:

MEA: 20 g·L⁻¹ malt extract (ME), 15 g·L⁻¹ agar-agar in demin. water.

SNL: 30 g·L⁻¹ glucose monohydrate, 15 g·L⁻¹ agar-agar, 4.5 g·L⁻¹ asparagine monohydrate, 3 g·L⁻¹ yeast extract, 1.5 g·L⁻¹ potassium dihydrogen phosphate, 0.5 g·L⁻¹ magnesium sulfate hydrate, 400 μg·L⁻¹ EDTA, 90 μg·L⁻¹ zinc(II) sulfate heptahydrate, 80 μg·L⁻¹ ferric chloride hexahydrate, 30 μg·L¹ manganese(II) sulfate monohydrate, 5 μg·L⁻¹ copper(II) sulfate pentahydrate, in demineralized water and adjusted to pH 6.0.

For cultivation, an approximately 0.5 cm² piece of mycelium from an 80% overgrown agar plate was placed on a new agar plate, the plate sealed with Parafilm® and incubated at 24° C. in the dark.

1.3 Screening on Blackcurrant Residue Streams

The cultivation of the fungi of the division Basidiomycota on the residue streams leaves and pomace of blackcurrant was carried out in submerged, emersed and fixed bed culture. Initially, the residue streams were used as the sole source of nutrients for the 26 fungi tested.

For the cultivation of the fungi in emersed culture, agar plates were prepared with substrate in different concentrations. Depending on the substrate, different concentrations of agar were required. These were inoculated with the 26 selected fungi (Table 1) in analogy to strain maintenance. After 4 to 7 d, depending on the growth of the fungi, the plates were examined for the first time for their sensory properties. To emersed culture of W. cocos on currant pomace, wine pomace, strawberry puree residue or raspberry puree residue for the further experiments, 3% pomace was added to 30% of demin. water and 1.5% or 3% agar in 70% demin. water was autoclaved and mixed before pouring. Each plate contained approximately 16 mL of medium.

For the cultivation of the fungi in submerged culture, precultures (100 mL) were first prepared in 2% ME medium. This was done by transferring an approx. 1 cm² piece of agar overgrown with mycelium from the strain maintenance plate into an Erlenmeyer flask containing ME medium, where it was homogenized using a disperser (Ultraturrax, 10,000 rpm, s). The preculture was incubated for 8 d at 24° C. in the absence of light at 150 rpm. For inoculation of the main culture, the preculture was homogenized (Ultraturrax, 10,000 rpm, s), centrifuged (3,000 g, 10 min), and the supernatant decanted. The mycelium was washed twice with sterile water and then suspended in 100 mL of sterile water. Of this suspension, 4 mL was added to 40 mL of main culture medium. The main culture medium consisted of 15 g·L⁻¹ or 30 g·L⁻¹ substrate in demineralized water. These cultures were grown at 24° C. in the dark at 150 rpm. Sensory analysis was performed from the 3rd day of culture by smelling directly on the flask. For comparison, a main culture in ME medium as well as non-inoculated main culture media were included as blank values.

For the cultivation of the fungi in fixed-bed culture, the precultures were cultivated and homogenized in the previously described manner. From this suspension, 2 mL was added to the fixed-bed culture medium. To prepare the fixed-bed culture medium, 20 g of pomace, 10 g of water, and 2 g of monosodium L-aspartate (Asp) were homogenized in a flask and autoclaved. The inoculated fixed-bed culture media were statically cultured at 24° C. in the dark at 150 rpm. For comparison, non-cultured fixed-bed media were included as blanks.

1.4 Optimization of Cultivation on Blackcurrant Residue Streams

To optimize the aroma profile and intensity, other sources of nutrients were added to the substrate. Besides adjusting the pH, glucose, trace elements (Mg²⁺, Zn²⁺, Fe³⁺, Mn⁺², Cu²⁺, K⁺, Cl⁻, SO₄²⁻, PO₄³⁻), monosodium L-aspartate and other amino acids were supplemented.

In addition, the substrate concentrations of the currant side streams were varied.

1.5 Sensory Analysis

Sensory analyses were carried out by a trained panel (n=4) to pre-select the fungus-substrate combinations. For this purpose, the cultures were examined in a “simple descriptive test” with evaluation of the overall impression on several culture days. The scale for the evaluation of the overall impression comprised six levels from very negative (0) to very positive (5). The identification of the aromas was done by GC-MS-O.

1.6 Determination of Mycelial Growth

During cultivation, the growth of the fungi was documented daily by drawing the overgrown area on the back of the agar plate and determining the diameter. From this, the overgrown mycelial area was calculated.

1.7 pH Value Determination

To determine the course of the pH value of the submerged cultures, an aliquot (2 mL) was taken and the pH was determined using a pH meter. For the emersed cultures, the pH of the cultures was determined by homogenizing the agar of one plate after addition of 15 mL of demin. water using Ultraturrax (10,000 rpm, 10 s), then centrifuging (10 min, 4,000 g) and measuring the pH in the supernatant using a pH meter.

1.8 GC-MS-O Analysis

The aromas of the emersed cultures on currant pomace were extracted by strbarsorptive extraction (SBSE) for 60 min at 24° C. For this purpose, 10 mm magnetic stir bars with 0.5 mm PDMS (polydimethylsiloxane) coating (Twister, GERSTEL, MOhlheim an der Ruhr, Germany) were fixed with a magnet in the headspace of the cultivation vessel. After incubation, the magnetic stir bars were removed with a magnetic rod, rinsed with demin. water, dried with a lint-free cloth and placed in a conditioned Thermal Desorption Unit (TDU) liner (GERSTEL). TDU desorption was performed without splitting. Analytes were cryofocused using a glass evaporator tube with silanized glass wadding (GERSTEL) in a Cold Injection System 4 (CIS) (GERSTEL). Gas chromatographic analysis was performed using GC-MS-O consisting of a 7890B GC (Agilent Technologies, Waldbronn, Germany) coupled to a 5977B MSD (Agilent Technologies) and an olfactometry detector port (ODP) (GERSTEL). An Agilent J&W VF-WAXms (30 m×0.25 mm ID×0.25 μm) was used as polar column, and an Agilent J&W DB-Sms (30 m×0.25 mm ID×=0.25 μm) was used as nonpolar separation phase. Helium was used as carrier gas at a constant flow rate of 1.56 mL·min⁻¹. The gas flow was directed into the MS and ODP with a 1:1 split. Further settings are listed below: Septum purge flow 3 mL·min⁻¹, scan mode total ion current (TIC), scan range m/z 33-300, ionization energy 70 eV, El source temperature 230° C., quadrupole temperature 150° C., MS transfer line temperature 250° C., He quench gas 2.25 mL·min⁻¹, N₂ collision gas 1.5 mL·min⁻¹, ODP 3 transfer line temperature 250° C., ODP mixing chamber temperature 150° C., ODP makeup gas N₂.

To investigate the changes in the relative intensities of the aromas and for identification using VF-WAXms, the oven temperature was increased after 3 min at 40° C. to 150° C. at 10° C.·min⁻¹, then to 240° C. at 20° C.·min⁻¹, and the temperature was maintained for 7 min. The TDU temperature program heated from 40° C. (0.5 min) to 250° C. (10 min) at 360° C.·min⁻¹. At the CIS, after a hold time of 0.5 min, the temperature was heated from −20° C. to 250° C. at 12° C.·s⁻¹ and held for 5 min. The CIS split ratio was varied between splitless and 50:1.

For semi-quantitative estimation and determination of retention indices using DB-5 ms, the oven was heated after 3 min at 40° C. to 320° C. at 5° C.·min⁻¹ and the temperature was maintained for 7 min. Differently, the TDU was heated to 250° C. at 120° C.·min-. Cryofocusing by CAS was differently performed at −100° C. The split ratio at the CIS was varied between splitless and 50:1.

Extraction of the aromas of the emersed cultures of W. cocos on wine pomace, strawberry puree residue or raspberry puree residue was carried out using headspace SBSE in the previously mentioned manner. Subsequent gas chromatographic analysis was performed using a GC-MS-FID consisting of an 8890 GC (Agilent Technologies) coupled to a 5977B MSD (Agilent Technologies) and a flame ionization detector. The column used was an Agilent VF-WAXms (30 m×0.25 mm ID×0.25 μm). The oven temperature was increased from 40° C. to 230° C. at 3° C.·min⁻¹ and held for 30 min. The TDU temperature program heated from 30° C. to 150° C. at 60° C.·min⁻¹ and was held for 10 min. The CIS was heated from −20° C. to 250° C. at 12° C.·s⁻¹ after a hold time of 0.1 min and the temperature was held for 10 min. The CIS split ratio used was 3:1. Helium was used as the carrier gas with a constant flow rate of 2.4 mL·min⁻¹. The gas flow was directed into the MS and FID with a split of 4:1. Other settings, which differ from previously mentioned systems, are listed below: Scan range m/z 25-370, MS transfer line temperature 280° C., FID H₂ flow 35 mL·min⁻¹, FID makeup gas N₂.

For the analysis of the fixed-bed cultures, 3 g of the fixed-bed culture medium grown with the mycelium was weighed into a 20 ml headspace vial and analyzed by solid phase micoextraction (SPME) in the headspace. For this purpose, the sample was incubated at 45° C. with 250 rpm for 20 min and extracted with a polydimethylsiloxane/divinylbenzene fiber for 5 min. Desorption was carried out splitless in a CIS 4 for 60 s at 230° C. Gas chromatographic analysis was performed using GC-MS consisting of a 7890B GC coupled to a 5977B MSD. An Agilent DB-FFAP (60 m×0.25 mm ID×0.25 μm) was used as the column. The oven temperature was increased after 5 min at 40° C. to 230° C. at 10° C.·min⁻¹ and the temperature was maintained for 10 min. Helium was used as the carrier gas with a constant flow rate of 1.5 mL·min⁻¹. The gas flow was directed into the MS and FID with a 10:1 split. Other deviating settings are listed below: Scan range m/z 25-550, MS transfer line temperature 250° C., FID H₂ flow 30 mL·min-, FID makeup gas N₂.

1.9 Identification of Aroma Substances

For the identification of the substances 10-30 mg of the reference substance were mixed with 50-1000 μL DMSO (Carl Roth, Karlsruhe Germany) depending on the solubility and if necessary filled up to 1 mL with demin. water. The following aroma substances were used for analysis: 2-aminobenzaldehyde ≥98% (Sigma Aldrich), 2-nonanone ≥99% (Acros Organics, Geel, Belgium), linalool 97% (Acros Organics), methyl anthranilate 99% (Acros Organics), (+)-2-octanol 98% (Alfa Aesar, Haverhill, USA), geraniol 97% (Alfa Aesar), 2-octanone 98% (Sigma Aldrich, St. Louis, USA), linalool oxide isomer mixture (Sigma Aldrich), 2-undecanone pure (Honeywell Fluka, Bucharest, Romania), benzaldehyde pure (AppliChem, Darmstadt, Germany). From these, 1:200 and 1:1000 dilutions were prepared with water and 0.5 mL of the solutions were plated out on agar (1.5% agar in demin. water). After one hour, a Twister® was attached to the headspace with a magnet and analyzed by GC-MS using the methods described in the previous point 1.8. Retention indices were calculated according to van den Dool and Kratz (van Den Dool. H.; Dec. Kratz, P., Journal of Chromatography A, (1963) 11, 463).

1.10 Semiquantitative Estimation of the Concentrations of Methyl Anthranilate, Linalool and Benzaldehyde

For semi-quantitative estimation, linalool, methyl anthranilate and benzaldehyde were dissolved in demin. water. For benzaldehyde, standards were prepared with concentrations of 3, 4, 8, 12, and 16 mg·L⁻¹, respectively; for methyl anthranilate, 9, 14, 19, 41, 80 mg·L⁻¹; and for linalool, 120, 241, 361, and 722 mg·L⁻¹, respectively. Of these, 0.5 mL each was plated out on an agar plate (Ø 10 cm, 16 mL 1.5% agar in demin. water). After one hour, a Twister® was attached to the headspace using a magnet and analyzed in analogy to the samples. By determining the peak areas calculated by Extracted Ion Chromatogram (EIC) of a fragment specific for the substance, the intensities detected in the sample were compared with the intensities of the standard substances and the samples were thus assigned to a concentration range.

2. Results

To investigate the biotransformation of blackcurrant side streams for the synthesis of natural aromas, a total of 26 fungi of the division Basidiomycota were tested. Table 2 gives an overview of the evaluation of the odor impressions of the tested fungus-substrate combinations in emersed and submerged culture.

TABLE 2
Sensory evaluation of the overall impression of the fungus-
substrate combinations between 0 (very negative) and 5
(very positive).

| | Emersed | Emersed | Submerged | Submerged |
|---|---|---|---|---|
| | culture | culture | culture | culture |
| Name | leaves | pomace | leaves | pomace |
| A. campestris | 2 | 1 | 4 | 1 |
| A. aegerita | 0 | 1 | 5 | 4 |
| A. melea | 0 | — | — | — |
| B. adusta | 2 | 2 | 2 | 2 |
| C. comatus | 0 | 1 | 5 | 5 |
| C. limbatus | 0 | — | — | — |
| F. velutipes | — | — | 1 | 1 |
| G. odoratum | 5 | 5 | 5 | 5 |
| H. fasciculare | 3 | — | 4 | 4 |
| I. consors | 3 | 2 | 2 | 2 |
| L. sulphureus | 5 | 0 | 4 | 1 |
| L. edodes | 3 | 0 | 0 | 0 |
| L. nuda | — | — | 1 | 0 |
| L. pyriforme | 1 | 1 | 0 | 4 |
| M. cohortalis | — | — | 0 | 0 |
| M. pseudocorticola | 5 | 5 | 5 | 0 |
| M. scorodonius | 5 | 0 | 5 | 5 |
| P. serotinus | — | — | 3 | 0 |
| P. chrysosporium | 0 | 0 | 3 | 1 |
| P. flabellatus | 0 | 1 | 5 | 5 |
| P. sapidus | 5 | 1 | 5 | 2 |
| S. crispa | 3 | 0 | 5 | 0 |
| S. hirsutum | 0 | 1 | 1 | 0 |
| T. suaveolens | 4 | 0 | 0 | 3 |
| T. chioneus | — | — | 3 | 1 |
| W. cocos | 5 | 5 | 5 | 5 |

—: cultivations not carried out.

Based on the sensory analysis, several interesting fungus-substrate combinations were identified. The fungi G. odoratum and W. cocos showed very intense odor impressions on all tested substrates in all tested cultivation types. G. odoratum produced here a citrus-like aroma, the aroma of W. cocos was floral, fruity and reminiscent of wild strawberries.

For a more detailed analysis of the growth behavior of W. cocos, the fungus was cultivated on different media. In addition to the pomace, malt extract agar was selected, which was well suited as a full medium for the growth of the fungus. An addition of monosodium Laspartate resulted in a particularly intense aroma when cultivated on pomace. In addition to the odors, the cultures differed both visually and by their growth (see FIG. 1).

With addition of Aspartat, both the mycelium and the nutrient medium turned brown. In addition, the growth was reduced compared to a reference medium (see FIG. 2). Less biomass and a very flat mycelium were formed.

With the addition of 0.624% monosodium L-aspartate, the pH value dropped less than in the cultures without aspartate (see FIG. 3). Both in the fermentation on pomace and on MEA medium, the pH value decreased over the course of the culture to about 2.5. In contrast, no drop in pH was observed in the medium from pomace with aspartate addition. In these cultures, the smell of wild strawberries was particularly intense.

For the analysis of the aroma composition, SBSE analyses were performed in combination with GC-MS-O. The fermentations of W. cocos on blackcurrant pomace with the addition of 0.624% monosodium L-aspartate were identified as particularly interesting. The detected aromas were significantly different from those detected in fermentations on MEA or currant pomace or currant leaves without the addition of aspartate.

In particular, C8 aromas such as 3-octanone, 1-octen-3-ol, 1-octanol and (E)-2-octen-1-ol were detected in the MEA medium (see FIG. 4). These are described as typical fungal aromas (Hofrichter, M., ed., Industrial Applications, (2011), Berlin, Heidelberg, Springer Berlin Heidelberg). This correlated well with the olfactory impression, which was described as fungal, acidic and smelling of tropical fruits.

In comparison, the aroma differed significantly when W. cocos was cultivated on currant pomace with aspartate. The culture began to smell floral and fresh after only a few days. Furthermore, no aromas reminiscent of fungus were perceptible. After about 10 days of culture, the smell intensified with floral notes and an intense fruity aroma reminiscent of wild strawberries.

The signal intensities of the aromas were significantly higher when cultivated on the aspartate-supplemented blackcurrant pomace than when cultivated on MEA (see FIG. 5). The intensities of linalool were particularly high, which correlated well with the floral odor of the culture.

In addition to linalool, further compounds were identified by GC-MS-O investigations as characteristic of the aroma of the fungus-substrate combination. In particular, methyl anthranilate showed an intensely fruity odor of wild strawberries.

The complexity of the aroma composition was also underlined by the olfactory identified compounds. For this purpose, emersed cultures of W. cocos were examined by GC-MS-O on defined culture days in order to detect particularly potent aromas (see FIG. 6).

In the olfactory evaluation of the culture thus carried out, the substances linalool and methyl anthranilate were very strongly perceptible. Benzaldehyde was also perceived with its characteristic odor of bitter almonds by means of ODP. In addition to these key aroma substances, a large number of other odors were perceptible, for which a substance classification is still pending.

Table 3 shows suggestions for selected substances contained in the sample with their retention indices (RI) according to van den Dool and Kratz and the odor compared with the retention indices of the standard substances and the odors described in the literature. The associated mass spectra (not shown) confirmed the formation of 2-octanone, 2-nonanone, linalool oxides, benzaldehyde, geraniol, 2-octanol, 2-aminobenzaldehyde, methylanthranilate and linalool by comparison with corresponding comparison spectra from the NIST database using samples from an emersed culture of W. cocos on 3% pomace+0.624% monosodium L-aspartate.

TABLE 3
Retention indices (RI) according to van den Dool and Kratz and odors of
the substances detected in the sample compared to standard substances (STD)
and odors described in the literature. VF-WAXms and DB-5ms denote the respective
separation phase used (see experimental part, point 1.8).

| | VF-WAXms | | DB-5ms | | Odor | |
|---|---|---|---|---|---|---|
| | Sample | STD | Sample | STD | GC-MS-O | Literature |
| 2-nonanone | 1393 | 1393 | 1093 | 1093 | green | fresh, sweetish, |
| | | | | | | green, grassy, |
| | | | | | | earthy ^{[6]} |
| 2-octanol | 1417 | 1417 | 1005 | 1004 | green | fresh, spicy, |
| | | | | | | green, woody, |
| | | | | | | herbaceous ^{[7]} |
| 2-octanone | 1290 | 1290 | 994 | 993 | green | earthy, herbaceous, |
| | | | | | | woody |
| Benzaldehyde | 1536 | 1536 | 968 | 967 | marzipan, | sweetish, almond, |
| | | | | | sweetish | cherry ^{[9]} |
| Geraniol | 1848 | 1848 | 1252 | 1252 | green | sweetish, flowery, |
| | | | | | | geranium, |
| | | | | | | fruity |
| Linalool | 1547 | 1547 | 1103 | 1102 | floral, fresh, | citrus, floral, |
| | | | | | citrus | sweetish, rose, |
| | | | | | | green, blueberry ^{[11]} |
| Linalool oxide I | 1446 | 1445 | 1075 | 1074 | green, fir, | floral, herbaceous, |
| | | | | | dull | |
| Linalool oxide II | 1474 | 1473 | 1090 | 1090 | dull | earthy, green ^{[12]} |
| 2-aminobenzaldehyde | 2176 | 2180 | 1222 | 1222 | sweetish, wild | sweetish, orange |
| | | | | | strawberry, | blossom, |
| | | | | | peach | fruity ^{[13]} |
| Methyl anthranilate | 2271 | 2269 | 1345 | 1345 | fruity, wild | fruity, grape, |
| | | | | | strawberry, | orange blossom ^{[14]} |
| | | | | | sweetish | |

^{[13]} Wood, William F.; Brownson, Mary; Smudde, R. Allen; Largent, David L. (1992): 2-Aminobenzaldehydes. The Source of the “Sweet Odor” of Hebeloma sacchari-olens. In: Mycologia 84 (6), p. 935. DOI: 10.2307/3760296.

The retention indices as well as the mass spectra and odors of the sample and standard were in good agreement.

Using headspace SBSE, the formation kinetics for the main aromas were recorded at intervals of 24 h over a period of 14 d (see FIG. 7).

Some aroma active compounds, such as benzaldehyde, linalool, linalool oxide I, II and 2-undecanone were formed almost exclusively on the medium of pomace and aspartate (3TT+Asp). Other compounds, such as 2-nonanone, geraniol and methyl anthranilate were formed both on the medium of pomace and aspartate and on the medium of MEA and aspartate (MEA+Asp).

The aroma analysis data (see FIGS. 8 and 9) correlated well with those of the sensory analysis, in which the characteristic odor was described as most intense on the 10th day of culture.

Linalool, methyl anthranilate and benzaldehyde were perceived as particularly characteristic of the aroma. For this reason, a semi-quantitative estimate was made of the aroma concentrations when W. cocos was cultivated on pomace with aspartate after 10 d (see FIG. 10).

Based on the calibration series, values of 95-135 μg per plate could be estimated for linalool, approx. 5 μg for benzaldehyde and 5-20 μg for methyl anthranilate. Considering the plate volume of about 16 mL, this corresponded to about 7,000±1,500 μg·L⁻¹ linalool, 350±50 μg·L⁻¹ benzaldehyde, and 700±350 μg·L⁻¹ methylanthranilate.

A sensory evaluation of the cultivation of W. cocos on conversion media based on blackcurrant pomace with and without the addition of aspartate was carried out by a trained panel. The emersed cultures with the addition of aspartate were rated as significantly more intense in terms of the attributes “fruity”, “flowery” and “wild berry” (see FIG. 11).

By cultivating W. cocos in conversion media with an ingredient based on plant parts of the grapevine family or rose family, very interesting aroma profiles could also be produced. Thus, the cultivation of W. cocos on media consisting of wine pomace of the varieties Müller-Thurgau, Gewürztraminer or Muscaris resulted in aroma profiles with fruity and floral notes reminiscent of wild strawberries, depending on the duration of cultivation (see Table 4).

TABLE 4
Sensory evaluation of a conversion on a culture medium according
to the invention using the example of an emersed fermentation of
W. cocos on 3% wine pomace of different varieties: MT: Müller-
Thurgau, GT: Gewürztraminer, MU: Muscaris. Summarized
odor attributes for different cultivation durations.

| Cultivation | | | |
|---|---|---|---|
| time (d) | MT | GT | MU |
| 5-6 | Grape juice, | Grape juice, | Grape juice, |
| | fruity, sweetish | fruity, sweetish | fruity, |
| | | | sweetish, green |
| 7-11 | Grape, acidic, | Grape, sourish, | Grape, acidic, |
| | fungal, fruity | fungal, sweetish | fungal, fruity |
| 12-18 | Curry powder, | Curry powder, | Curry powder, |
| | spicy, | spicy, fungal, old, | spicy, fruity, |
| | fruity, dry | dry | fungal, dry |
| 19-28 | spicy, floral, fruity, | sweetish, fruity, | spicy, fungal, |
| | wild strawberry, | fungal, | roasted, wild |
| | citrus, roasted | wild strawberry, | strawberry |
| | | musty | |
| 29-47 | wild strawberry, | wild strawberry, | wild strawberry, |
| | sweetish, fruity, | sweetish, fruity, | sweetish, fruity, |
| | fungal | flowery | dry, floral |

s A sensory evaluation of the emersed cultures of W. cocos on conversion media based on grape pomace of the Müller-Thurgau variety with the addition of aspartate showed similarities with emersed cultures based on redcurrant pomace (see FIG. 12).

By means of gas chromatographic analysis, the aroma-active compounds 2-octanone, 2-nonanone, 2-undecanone, linalool oxides, benzaldehyde, geraniol, methyl anthranilate and linalool were detected in cultures of W. cocos on media containing ingredients of the rose or grapevine family. Exemplary chromatograms of emersed cultures of W. cocos on Muscaris wine pomace, strawberry puree residue or raspberry puree residue, each with the addition of 0.6% monosodium L-aspartate, are shown in FIGS. 13, 14 and 15.

A comparison of the amounts of aroma-active compounds formed when W. cocos was grown in emersion on media containing ingredients of the rose family or grapevine family with additional variation of the glucose content is shown in FIG. 16. The aroma-active compounds shown could not be detected in the non-cultivated reference media without added glucose (M1 (0 d)), or only in significantly low amounts. The cultivation on the media based on strawberry and raspberry puree residues, which additionally contained 0.4% and 0.8% glucose (M2 and M3, respectively), resulted in lower formation amounts of the presented aroma-active compounds compared to medium M1 (see FIG. 16). The formation of the presented aroma-active compounds therefore resulted primarily from the biotechnological conversion of the plant ingredients present in the medium.

The cultivation methods used had a significant influence on the quantitative composition of the aroma of the cultures. Thus, the aroma-active compounds, which were characteristic for the aroma of the emersed cultures of W. cocos, were also detected in deviating amounts by submerged cultivation or fixed-bed cultivation of the fungus. An exemplary chromatogram of a fixed-bed cultivation of W. cocos on a medium with strawberry puree residues and aspartate is shown in FIG. 17.

1. A method for producing an aroma substance or a mixture of aroma substances, comprising the steps:
providing a conversion medium comprising a plant component of the gooseberry family, the rose family and/or the grapevine family;
contacting the conversion medium with at least one fungus from the division of stander fungi capable of forming an aroma substance or a mixture of aroma substances on the conversion medium;
converting the plant component to the aroma substance or mixture of aroma substances with the aid of the fungus; and optionally
recovering the aroma substance or mixture of aroma substances.

2. The method of claim 1, wherein
a) the gooseberry family is selected from the group of species of redcurrants, gooseberries and their hybrids,
b) the rose family is selected from the group of species of strawberry, raspberry and their cultivars and hybrids, and/or
c) the grapevine family is selected from the group of species of grapevines and their hybrids.

3. The method of claim 1, wherein the plant component is selected from the group consisting of leaves, buds, leaf buds, flower buds and berry fruits, and their sub-components, extracts and pomace.

4. The method of claim 1, wherein the at least one fungus from the division of the stander fungi is selected from the group consisting of A. campestris, A. aegerita, A. melea, B. adusta, C. comatus, C. limbatus, F. velutipes, G. odoratum, H. fasciculare, I. consors, L. sulphureus, L. edodes, L. nuda, L. pyriforme, M. cohortalis, M. pseudocorticola, M. scorodonius, P. serotinus, P. chrysosporium, P. flabellatus, P. sapidus, S. crispa, S. hirsutum, T. suaveolens, T. chioneus and W. cocos.

5. The method of claim 4, wherein the fungus is W. cocos or G. odoratum.

6. The method of claim 1, wherein the aroma or mixture of aromas comprise at least one compound selected from the group consisting of 2-octanone, 2-nonanone, 2-undecanone, linalool oxides, benzaldehyde, geraniol, 2-octanol, methyl anthranilate, 2-aminobenzaldehyde and linalool.

7. The method of claim 1, wherein the mixture of aroma substances comprises at least 2 compounds selected from the group consisting of linalool, benzaldehyde and methyl anthranilate.

8. The method of claim 1, wherein the conversion medium provided comprises an aspartate source.

9. The method of claim 1, wherein the pH of the conversion medium during the conversion is within a range of pH 2 to pH 7.

10. The method of claim 1, wherein the conversion is carried out in emersed culture, fixed bed culture or submerged culture.

11. (canceled)

12. A composition prepared according to the method of claim 1, comprising at least 2 compounds selected from the group consisting of linalool, benzaldehyde and methyl anthranilate,
in a weight by volume ratio of linalool to benzaldehyde of between 100:1 and 4:1 and/or
in a weight by volume ratio of linalool to methyl anthranilate of between 50:1 and 2:1, and/or
in a weight by volume ratio of benzaldehyde to methyl anthranilate of between 3:1 and 1:10.

13. The composition of claim 12, further comprising at least one other compound selected from the group consisting of 2-octanone, 2-nonanone, 2-undecanone, linalool oxides, benzaldehyde, geraniol, 2-octanol, methyl anthranilate, and linalool.

14. A nutritional, cosmetic, hygienic or edible preparation comprising of a composition of claim 12.

15. (canceled)

16. The method of claim 2, wherein the gooseberry family is selected from the group of species of white or red gooseberries, red, white, black or deep black currants, and their hybrids and varieties.

17. The method of claim 2, wherein the grapevine family is selected from the group of species of noble vines and their hybrids and varieties.

18. The method of claim 3, wherein the plant component is selected from the group consisting of leaves and berry fruits, and their sub-components, extracts and pomace.

19. The method of claim 7, wherein the mixture of aroma substances comprises linalool, benzaldehyde and methyl anthranilate.

20. The method of claim 8, wherein the aspartate source is from 0.1% to 20.0% based on the total weight of the conversion medium.

21. The method of claim 9, wherein the pH of the conversion medium during the conversion is within a range of pH 4 to pH 6.

22. The method of claim 21, wherein the pH of the conversion medium during the conversion is within a range of pH 4.2 to pH 5.5.
